(19) United States
(12) Patent Application Publication
(10) Pub. No.: US 2019/0231701 A1
(43) Pub. Date: Aug. 1, 2019

(54) Fill formulation method for hard, soft, and liquid capsules comprising the mixture of a polymer and a fill component that will migrate into or through a capsule shell with serviceable functions

(71) Applicant: Connor Francis Leach, Las Vegas, NV (US)

(72) Inventor: Connor Francis Leach, Las Vegas, NV (US)

(73) Assignee: Pileus LLC, Las Vegas, NV (US)

(21) Appl. No.: 16/376,874

(22) Filed: Apr. 5, 2019

Related U.S. Application Data
(63) Continuation of application No. 15/438,898, filed on Feb. 22, 2017.
(60) Provisional application No. 62/442,458, filed on Jan. 5, 2017.

(30) Foreign Application Priority Data
Aug. 7, 2017 (GB) GB1712608.7

(51) Int. Cl. A61K 9/48

(52) U.S. Cl. CPC A61K 9/4825

(57) ABSTRACT

The present disclosure described is a fill formulation method for hard, soft, and liquid capsules comprising a polymer and a fill component, wherein the polymer comprised of mycoprotein is mixed with a fill component mixture comprised of a plasticizing agent, a preservative agent, a lubricant agent, and purified water, which will migrate into or through a capsule shell, with attributes of serviceable functions.

### FIELD OF INVENTION

The present invention relates to improved forms of medicine in the pharmaceutical industry for medical usage and improved forms of supplements in the non-pharmaceutical industry for dietary usage.

### ISSUES

There are two types of capsules on the market, gelatin and hydroxypropyl methylcellulose (HPMC), which are animal and plant based capsules that both have problematic issues.

Gelatin capsules are derived from genetically modified productions of animal connective tissues containing animal skin and crushed animal bones, and are subsequently made from boiled cattle, pigs, and fish that are slaughtered and hazardously transferred from point A to point B. Gelatin capsules have an exposed weakness to averagely humid temperatures and begin dissolving at 25° C. and begin melting at 40° C.

Hydroxypropyl methylcellulose (HPMC) capsules are strictly made from pollen and pine, thus with 18% of humans allergic to pollen and 2% of humans allergic to pine, an average 19% sum of humans have allergens to the alternative vegetarian capsules, which do not digest in the human body. Cellulose is not an essential nutrient and assists with impelling food forcefully through the digestive system, processing fermentation, causing gas in the large intestine from intestinal bacteria. Hypromellose, short for hydroxypropyl methylcellulose, melts and dissolves at 260° C. forever making its chemical formula humanly impossible to digest. Cellulose is very expensive, which is why hypromellose is genetically modified in its capsule manufacturing, thus perceiving the vegetarian alternative to be a greater health risk than hazardous gelatin capsules.

A hypromellose capsule is three times the price of a gelatin capsule, and the most common cause of all allergic reactions to oral vaccinations identify gelatin as the allergen.

### BACKGROUND

The present invention described provides a solution to the issues of gelatin capsules and hypromellose capsules from alternative mycoprotein capsules.

### BRIEF DESCRIPTION OF INVENTION

Mycoprotein is known as fungal protein, first produced in the UK during 1980, eleven years after Fungi was named as its own Kingdom. Myco is the greek word for Fungus. Fungi-Culture is the process of producing fungi in medicine. Myco is made up of chitin, glucans, and protein. Myco relates to humans as a more similar life form than plants, through storing food as glycogen, the same way an animal cell stores glucose. Unlike gelatin, mycoprotein has dietary fiber with significantly less lipids. Mycoprotein has a lower protein mass than gelatin, which holds a higher quality of protein than beef. Mycoprotein is naturally low in fats and saturates with zero cholesterol, zero calories, and zero mycotoxins. Mycoprotein is Non-GMO, unlike gelatin and hypromellose, therefore mycoprotein is not genetically modified and is fundamentally vegetarian, and essentially vegan. All of which identifies the basic makeup of the mycoprotein capsule invention as a preferred digestive method. Mycoprotein is a strain of fungi, consisting of 44% protein, scientifically defined as Fusarium venenatum A3/5 and Fusarium venenatum PTA-2684; organic and non-organic. Mycoprotein, when grown properly, has the potential to double its mass every five hours, which speeds the capsule manufacturing process.

The chemical formula of mycoprotein is C25H42N6O3. Both mycoprotein strains, Fusarium venenatum A3/5 and Fusarium venenatum PTA-2684, are used within the fill formulation as a polymer to migrate into or through a capsule shell.

The invention described presents itself as a hard, soft, and liquid capsule, which are banded and or sealed on the exterior surface with pullulan, or left to remain non-sealed.

The aqueous solution of the fill formulation mixture is made up of a purified aqueous solvent (H2O), sodium lauryl sulfate used as a lubricant agent, and sulfur dioxide used as a preservative agent. The mixture of purified H2O at 20° C., sodium lauryl sulfate, and sulfur dioxide, all comprise the aqueous solution with a viscosity level ranging from 1 to 100 cPs. The aqueous solution is then mixed with a plasticizing agent, which is typically glycerol. The mixture of the aqueous solution and plasticizing agent comprise the fill component with a viscosity level ranging from 800 to 1000 cPs. The fill component is mixed with the mycoprotein polymer, Fusarium venenatum A3/5 and or Fusarium venenatum PTA-2684, which comprises the aqueous composition with a viscosity level ranging from 1500 to 3000 cPs. The entire fill formulation method comprising the aqueous composition mixture will migrate into or through a capsule shell. The dried composition of the mycoprotein capsule shell comprises a mycoprotein polymer, a glycerol plasticizer, excipients from solutions, and a purified H2O solvent, in weight percentage.

TABLE 1
Non-Patent Document References

| Document No. | Author | Field | Year |
|---|---|---|---|
| 58:421-427 | Wiebe et al | Appl. Microbiol. Biotechnol., | (2002) |
| ISSN-0975-1491 | Krishna Vamshi | Colorant - The Cosmetics For | (2011) |
| | Allam, Gannu | The Pharmaceutical Dosage | |
| | Praveen Kumar | Forms | |
| US-20140227357-A1 | Jan Vertommen | Fill formulations claiming only to | (2014) |
| | | gelatin and cellulose polymers; | |
| | | listing mycoprotein as a taggant to | |
| | | reduce volatility as a liquid GRAS | |

### DESCRIPTION OF THE DRAWINGS

FIG. 1: is a graphical representation of a mushroom, which presents itself as the most identifiably basic conception of fungi. The mycoprotein polymer within the fill formulation mixture to produce mycoprotein capsules is based upon fungi, and or fungus, which is defined in latin as myco. The identifying letter (a) identifies the entirety of figure one.

FIG. 2: is a graphical representation of the chemical formula of mycoprotein, Fusarium venenatum A3/5 and or Fusarium venenatum PTA-2684, which are both strains of mycoprotein used as the polymer within the fill formulation mixture to produce mycoprotein capsules. The identifying letter (b) identifies the entirety of figure two.

FIG. 3: is a graphical representation of a two-piece mycoprotein capsule sealed and or banded with an exterior plasticizing constituent. In figure three, the identifying letter (c) identifies the body of the capsule, and the identifying letter (d) identifies the cap of the capsule.

FIG. 4: is a graphical representation of a two-piece, non-sealed, mycoprotein capsule that is also identified with the letter (c) identifying the body of the capsule shell, and is also identified with the letter (d) identifying the cap of the capsule shell.

FIG. 5: MYCOCAPS® by Pileus LLC, size XXX-V, (000-5).

FIG. 6: MYCOGELS® by Pileus LLC, size XXX-IV, (000-4).

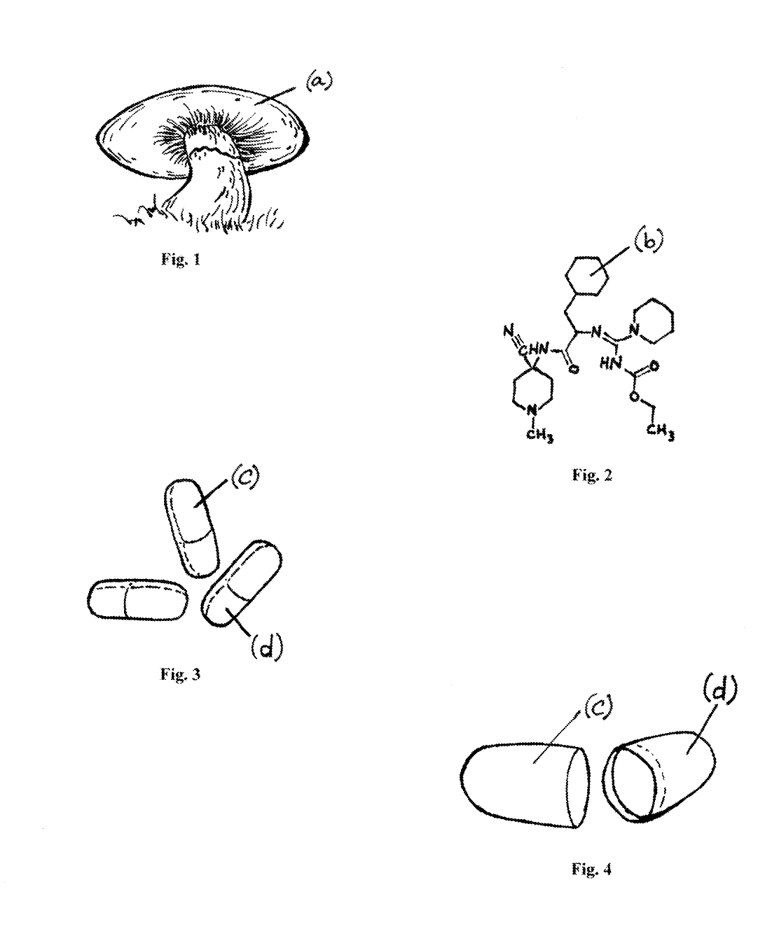

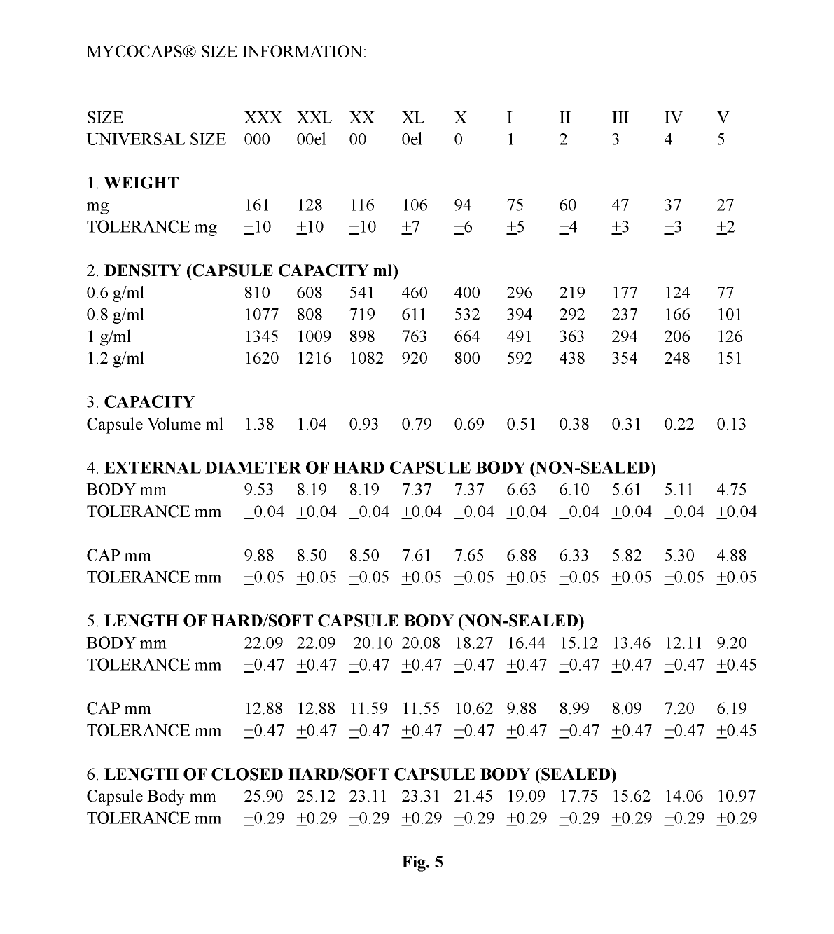

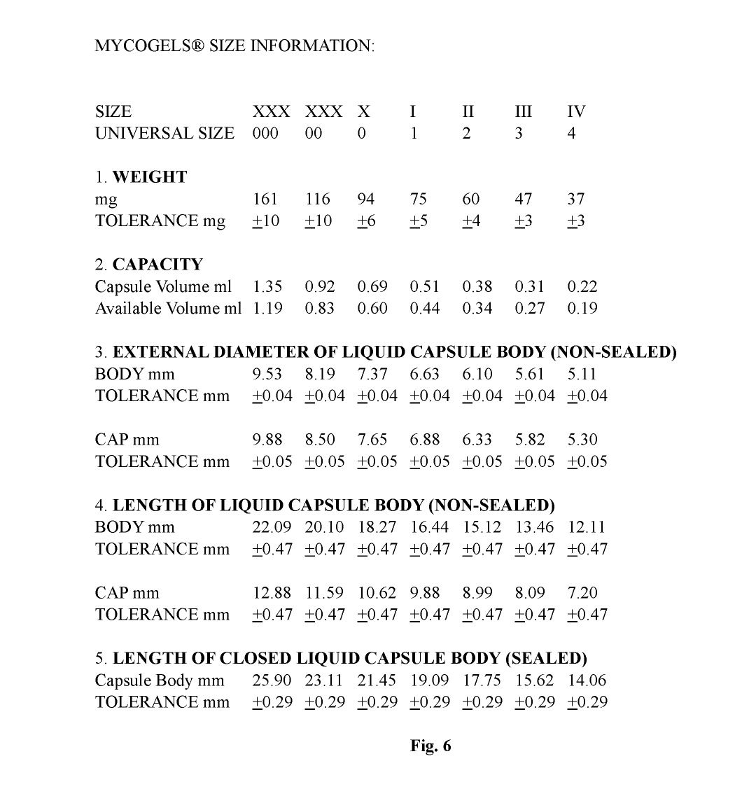

### DETAILED DESCRIPTION OF INVENTION:

The invention presented is a fill formulation mixture of a polymer, a plasticizing agent, and an aqueous solution.

The polymer selected for within the fill formulation mixture is mycoprotein. The two strains of mycoprotein used in the fill formulation mixture are Fusarium venenatum A3/5 and or Fusarium venenatum PTA-2684; organic and non-organic.

The plasticizer typically used is glycerol. Other plasticizing agents that can be used optionally within the fill matrix include acetyl tributyl citrate, acetyl triethyl citrate, acetylated monoglyceride, castor oil, coconut oil, dibutyl phthalate, dibutyl sebacate, diethyl phthalate, fructose, glucose, glucose syrup, glycerin, glycerine, non-crystalising solutions of sorbitol, polyethylene glycol (PEG 200-6000), polyglycerol, 2-propylene, propylene glycol, pullulan, sorbitol, triacetyl glycerine, tributyl citrate, triethyl citrate, xylitol, and mixtures thereof.

The aqueous solution is a mixture made up of a purified aqueous solvent (H2O), sulfur dioxide used as a preservative agent, and sodium lauryl sulfate used as a lubricant agent. The sodium lauryl sulfate has a pH of 7.5 to 8.5, and the plasticizing agent that is typically glycerol has a pH of about 7. Other substitutes for usage as preservative agents include benzoic acid, choline chloride, metal propyl esters of para hydroxy-benzoic, organic acid sulfur dioxide, propane acid, silicone dioxide, sodium bisulfite, sodium chloride, sodium metabisulfite, sorbic acid, and mixtures thereof. Other substitutes for usage as lubricant agents include 2-butanol, hydroxypropyl-β-cyclodextrin, L-carvone, methyl-2-pentanone, methyl ethyl ketone, methyl n-propyl ketone, 2-pentanol, 2-propanol, sodium coco-sulfate, and mixtures thereof

The mixture of the fill formulation process is as follows: As used herein, “v/v %” means by volume as percentage of total volume.

The aqueous solution comprises an aqueous solvent mixed with excipients. The aqueous solvent is comprised of purified water (H2O), and the excipients are fillers comprised of preservatives, lubricants, flavors, colors, an opacifier, and additional gelling agents.

First, the purified aqueous solvent is mixed at 94% to 97% (v/v), with sulfur dioxide at 2% to 4% (v/v), and sodium lauryl sulfate at 1% to 2% (v/v).

The flavor is added, or not added, as an excipient to the fill formulation method of the aqueous solution with sugar. In such case, as an additional additive to the fill formulation, the sugar mixed into the completed aqueous solution is 0.2% to 2.5% (v/v). Other substitutes of sugars for potential usage include agave nectar, arabinose, barbados sugar, barley malt syrup, barley malt, barley sugar, beet sugar, birch syrup, brown sugar, buttered syrup, cane sugar, caramel, carob syrup, castor sugar, coconut sugar, confectioner's sugar, corn syrup, date sugar, dehydrated cane juice, demerara sugar, dextrin, dextrose, disaccharide, evaporated cane juice, free sugar, fructose, fruit juice, fucose, galactose, glucose, golden syrup, granulated sugar, grape sugar, high fructose corn syrup, high maltose corn syrup, honey, icing sugar, inositol, inverted sugar syrup, jaggery lactose, malt extract, maltose, maltodextrin, mannose, maple sugar, molasses from sugar beats, molasses from sugar cane, monosaccharide, muscovado, non-centrifugal cane sugar, palm sugar, panocha, powdered sugar, raw sugar, refiner's sugar, ribose, rice syrup, rhamnose, saccharose, sorghum syrup, sucrose, sugarcane, sweet sorghum, syrup, toffee, treacle, trehalose, xylitol, xylose, yellow sugar, and mixtures thereof.

The coloring is added, or not added, as an excipient to the fill formulation method of the aqueous solution with dye. The coloring additive dye and or pigment is mixed into the aqueous solution with a concentration level ranging from 0.001% to 1.5% (v/v). Another process for dying the capsules is by performing a manual dipping and drying method of the exterior capsule shell into and out of a dye mixture.

The organic substitute for implementing colored dye into the aqueous solution is comprised of fruits and vegetables, which are raspberries for red coloring, mangos for yellow coloring, blackberries for purple coloring, carrots for orange coloring (beta-carotene), spinach for green coloring, and a combination of cabbage with added baking soda for blue coloring. All of which are organic food coloring agents used as additive dye within the aqueous solution.

Artificially synthetic mineral dyes are added, or not added, to the the aqueous solution, primarily FD&C or D&C colorants. These FDA approved colorants include; FD&C: Red #3 Erythrosine, Red #40 Allura Red, Yellow #5 Tartrazine, Yellow #6 Sunset Yellow, Blue #1 Brilliant Blue, and Blue #2 Indigo Carmine. D&C: Red #4, Red #17, Red #22, Red #27, Red #28, Red #33, Red #39, Orange #4, Yellow #7, Yellow #8, Yellow #10, Yellow #11, Green #5, Green #6, and Green #8. All of which are artificial coloring agents used as additive dye within the aqueous solution.

The opacifier is added, or not added, to the mixture of the aqueous solution to create an opaquely nontransparent concentration from titanium dioxide used at 0.25% to 5% (v/v).

Additional gelling agents are added, or not added, such as calcium, magnesium, and potassium to form a more sufficient gel-like aqueous solution at 0.01% to 0.05% (v/v).

The aqueous solution comprises a viscosity level of 1 to 100 cPs.

Second, the aqueous solution is mixed at 50% to 60% (v/v) with the plasticizing agent, typically glycerol, between 40% to 50% (v/v) comprising the fill component.

The fill component comprises a viscosity level of 800 to 1000 cPs.

As used herein, “w/v %” means by volume as a percentage of the total weight.

Third, the fill component at 35% to 70% (w/v) is mixed with the selected mycoprotein polymer at 30% to 65% (w/v) ultimately comprising the aqueous composition.

The aqueous composition comprises a viscosity level of 1500 to 3000 cPs.

The mycoprotein polymer within the aqueous composition for hard capsules makes up around 56% (w/v) with its fill component making up around 44% (w/v).

The mycoprotein polymer within the aqueous composition for soft capsules makes up around 43% (w/v) with its fill component making up around 57% (w/v).

The mycoprotein polymer within the aqueous composition for liquid capsules makes up around 30% (w/v) with its fill component making up around 70% (w/v).

As used herein, “w/w %” means by weight as a percentage of the total weight.

The composition of the dry capsule shell comprises mycoprotein polymer at 30% to 56% (w/w), a plasticizer, typically glycerol, at 20% to 30% (w/w), purified water at 24% to 40% (w/w), and a solution of various excipients ranging from 0.5% to 1.5% (w/w).

The development of the mycoprotein capsule shell is sealed, or left to remain non-sealed. When the mycoprotein capsule is sealed during its manufacturing, the capsule seals, encloses, holds, caters, harnesses, and preserves the drugs, medicine, supplements, and lack thereof, that are filled into the capsule shell for the preservative function of the mycoprotein capsules.

The representative placebo formulations, pharmaceutical dosages, therapeutic drugs, nutritional agents, and or dietary supplements for this function of mycoprotein capsules include, but are not limited to: Ace-inhibitors; acetaminophen; acetylsalicylic acid and its buffered form; acne drugs; albuterol and its salts; alkaline phosphatase; alkaloids; allantoin; aloe; aluminum acetate, amino acids; amino acid preparations; aminobenzoic acid; amoxicillin; ampicillin; amsalog; anabolic drugs; analgesics; anesthetics; anethole; angiogenesis inhibitors; amsacrine; antacids; antianginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; antinauseants; anti-stroke agents; anti-thyroid preparations; anti-tumor drugs; antitussives; anti-uricernic drugs; anti-viral agents; antiarthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; ascorbic acid; aspartame; aspirin; atenolol; bacitracin; beclomethasone; benzocaine; benzoic acid; benzophenones; beta blocking agents; betamethasone dipropionate; bethanechol; biotin; bisacodyl; bornyl acetate; botanical substances, brompheniramine maleate; bronchodilators; buspirone; caffeine; calcium carbonate; camphor; captopril; caseinate; cascara sagrada; castor oil; carbonate chlorohydrate; cardiovascular agents; carmustine; cefaclor, cefadroxil; cephalexin; cerebral dilators; cetyl alcohol; cetylpyridinium chloride; chelated minerals; chelating agents; chemotherapeutic agents; chloramphenicol; chlorcyclizine hydrochloride and pseudoephedrine; chlorhexidine gluconate; chloropentostatin; chloroxylenol; chlorpheniramine maleate; cholecystokinin antagonists; cholestyramine resin; choline bitartrate; chondrogenic; cinnamedrine hydrochloride; cimetidine hydrochloride; ciprofloxacin; citalopram; citrate; citric acid; clonidine; clofibrate; cod liver oil; codeine; codeine phosphate; cognition activators; contraceptives; coronary dilators; cortisone acetate; cough suppressants; cyanocobalamin; cyclizine hydrochloride; danthron; decongestants; deodorants; dermatological agents; dexbrompheniramine maleate; dextromethorphan hydrobromide; diabetes agents; diazepam; dibucaine; diclofenac sodium; digoxin; diltiazem; dimethicone; dioxybenzone; diphenhydramine citrate; diphenhydramine hydrochloride; diuretics; docusate calcium, doxycycline hyclate; doxylamine succinate; efaroxan; emollients; enalapril; enoxacin; enzymes; ephedrine; epinephrine bitartrate; erythromycin; erythropoietic drugs; erythropoietin; estropipate; ethinylestradiol; eucalyptol; expectorants; ferrous fumarate; fertility agents; fluoride; 5-fluorouracil; fluoxetine; folic acid; fosphenytoin; fungicides; furosemide; gabapentin; gastro-intestinal agents; gemfibrozil; gentamicin; glipizide; gluconate; glycerin; glyceryl stearate; griseofulvin; growth hormone; growth regulators; guaifenesin; hexylresorcinol; hormone replacement agents; hydrocodone bitartrate; hydrocortisone; hydrochloride salt; hydrochlorothiazide; hydroxide; hydroxide-alprazolam; 8-hydroxyquinoline sulfate; hyperglycemic agents; hypnotics; hypoglycemic agents; ibuprofen; indomethacin; inositol; insulin; iodine; ipecac; iron; isoxicam; ketamine; lactic acid; laxatives; lecithin; leuprolide acetate; LHRH (luteinizing hormone releasing hormone); lidocaine; lisinopril; liotrix; lovastatin; luteinizing hormone; trisilicate; magnesium carbonate, salicylate, and hydroxide; meclofenamate sodium; meclofenamic acid; medroxyprogesterone acetate; mefenamic acid; menthol; meperidine hydrochloride; metaproterenol sulfate; methenamine mandelate; methsuximide; methyl nicotinate; methyl salicylate; metoprolol tartrate; metronidazole; miconazole nitrate; migraine treatments; mineral supplements; minoxidil; morphine; mucolytics; naproxen; narcotics; neomycin sulfate; neuromuscular drugs; neuroleptics; niacin; niacinamide; nicotinamide; nicotine; nifedipine; nitroglycerin; nonoxynol-9; non-steroidal anti-inflammatory drugs; norethindrone; nutritional additives; nystatin; omega-3 polyunsaturated fatty acids; omeprazole; oxolinic acid; oxtriphylline; oxybenzone; padimate 0; para-aminobenzoic acid; paramethadione; pentaerythritol tetranitrate; pentostatin; pentobarbital sodium; peppermint oil; peripheral vasodilators; phenelzine sulfate; pheniramine maleate; phenobarbital; phenol; phenolphthalein; phenylephrine hydrochloride; phenytoin; phenylpropanolamine; pirmenol; piroxicam; polymyxin B sulfate; polypeptides; potassium; potassium chloride, nitrate, and inorganic salts; pramiracetim; pramoxine; prazepam; procainamide hydrochloride; procaterol; propoxyphene; propoxyphene napsylate; propranolol hydrochloride; prostaglandins; pseudoephedrine; psychotropics; pyridoxine; quinapril; quinestrol; quinidine gluconate; ralitoline; ranitidine; renin inhibitors; resorcinol; respiratory stimulants; riboflavin; salicylic acid; sesame oil; shark liver oil; simethicone; sodium; sodium bicarbonate; sodium monofluorophosphate; steroids; stimulants; sucralfate; sulfamethoxazole; sulfasalazine; sulfate; sulfur; sympatholytics; tacrine; terfenadine; tetracycline hydrochloride; theophylline; thioperidone; thyroid preparations; timolol maleate; tolmetin; tolnaftate; tranquilizers; tretinoin; triazolam; triclosan; trimetrexate; triprolidine hydrochloride; undecylenic acid; uterine relaxants; vaginal preparations; vancomycin; vasoconstrictors; verapamil hydrochloride; vertigo agents; vidarabine phosphate; vitamins; vitamins A, B, C, D, B1, B2, B6, B12, E, K; witch hazel; wound healing agents; xylometazoline hydrochloride; zinc; zinc sulfate; and zinc undecylenate.

Machines for stamping, labeling, and printing on the outer surface of the capsule shell are used, or not used, to determine the categorization of mycoprotein capsules with specific numbers and letters by portraying logos, names, symbols, and all forms of text in different fonts, designs, and colored text. Organic forms of ink used for stamping, labeling, and printing on mycoprotein capsules are used from dark fruits, and non-organic labels are processed from food coloring ink.

The active agents within the mycoprotein capsules include, but are not limited to: Enzyme inhibitors, enzymes, glycoproteins, hormones, lipoproteins, organic and or non-organic pharmaceutical proteins, polynucleotides, polypeptides, polysaccharides, and nucleoproteins. The animals that benefit from the effective agents in the mycoprotein capsules include, but are not limited to: Domesticated animals, farm animals, warm blooded mammals, and wild animals.

TABLE 2
Expired Patents Related to Improving Capsule Integrity

| U.S. Pat. No. | Inventors | Invention | Year |
|---|---|---|---|
| 2,566,645 | Weber Anton Philip, Weber | Method and means for | (1947) |
| | Frank Anton | making seamless capsules | |
| 2,624,163 | Frank E Stirn | Method of making colored | (1948) |
| | | gelatin capsules | |
| 3,413,396 | Stearns Carl Louis | Pigment marking gelatin capsules | (1963) |
| 4,591,475 | Ivan Tomka, Fritz Wittwer | Method for molding capsules | (1983) |
| 6,337,045 | Ralph R. Grosswald, Jeffory | Method for the manufacture of | (1995) |
| | B. Anderson, Clair S. And. | pharmaceutical cellulose capsules | |
| 6,346,231 | Joar Opheim | Flavored gelatin capsules and | (1999) |
| | | method of manufacture | |

### How it is Made:

The fill formulation mixture of the mycoprotein polymer, the plasticizing agent, and the aqueous solution comprise the aqueous composition.

The aqueous composition is prepared for capsule manufacturing to process mycoprotein capsules through being heated above melting temperatures of 80° C. for the allowance of the fill formulation to properly mix.

The aqueous composition is cooled down to gelling temperatures between 10° C. and 30° C. with a viscosity concentration level of 1500 to 3000 cPs. Any remaining unused mixture for production of the aqueous composition is reheated and held in holding tanks at 60° C., between molding processes, to prevent premature settling.

Dipping pins are preheated at 55° C. to 95° C. to be dipped into the aqueous composition where a film is obtained on the dipping pins after being removed from the aqueous composition. The dipping pins are subject to a relative humidity of 25% to 55%.

The aqueous composition film is dried on the dipping pins above gelling temperatures for the aqueous composition film to obtain molded shapes of capsule shells from the dipping pins.

A capsule manufacturing machine continues the process of development by dipping, spinning, and drying the aqueous composition. The capsule shells are removed from the pins and set aside onto a collet holding device for proper drying purposes, where knives trim the capsules of excess materials for the capsules to reach their appropriate shape, and all of the excess materials are sorted into a recycling. Machine tests are run for any capsules that are deemed to be defective from a dent of a pinch through an automatic detection scanning and sorting process where the defective capsules are removed with a vacuum and transferred into recycling bins.

Lastly, an exterior plasticizing process is performed by joining the capsule cap and body together by using a yeast film-forming agent, typically pullulan, and or other plasticizing agents such as glycerol, propylene glycol, 2-propylene, and mixtures thereof.

### How it Works:

MYCOCAPS® are hard and soft capsules that work suitably in temperatures ranging from 5° C. to 45° C. with functions of inhabiting, preserving, and digesting.

MYCOGELS® are liquid capsules that work suitably in temperatures ranging from 10° C. to 40° C., and are sufficiently suitable at 30° C., with shell durability in up to 75% humidity, and up 37° C. before moisture absorption, with functions of inhabiting, preserving, and digesting.

Mycoprotein capsules work through digesting in the human body and digesting in the animal body. Therefore, the invention described works to hold medicine, herbs, vitamins, health supplements, prescription drugs, dietary supplements, nutraceuticals, veterinary products, cosmetic products, and foodstuffs for breaking down in the digestive system. The invention described will work to aid as a pharmaceutical capsule in the medical field and work to aid as a non-pharmaceutical capsule for dietary supplement usage.

The first working function of the mycoprotein capsule shell is for catering to the storage of medicine, drugs, supplements, vitamins, and lack thereof.

The second working function of the mycoprotein capsule is to dissolve in the human and animal body with application to the digestive system for releasing medicine, drugs, supplements, vitamins, and lack thereof.

The third working function of the mycoprotein capsule is to inhabit sustainable temperatures for workability prior to digestion, while storing medicine, drugs, supplements, vitamins, and lack thereof.

### Conclusive Embodiments:

Alternative embodiments to the fill formulation of the disclosed invention for how it is made can be accomplished without appropriate measurements of the aqueous composition. For example, the fill formulation can be made up of 50% mycoprotein and 50% purified water as a means to create the mycoprotein capsules, regardless of workability and imperfections, without lubricant and preservative functions, which would ultimately enable the success of molding from gelling agents and plasticizing agents.

Another alternative embodiment to the fill formulation of the disclosed invention for how it is made can be by dividing the mycoprotein, plasticizer, and water into any such thirds of 100% volume of the completed aqueous composition, or by dividing the mycoprotein, plasticizer, and water into any such thirds of 100% weight of the aqueous composition.

The fill formulation comprising the aqueous composition for the disclosed invention of how the mycoprotein capsules are made is approved by the FDA and is not regulated by the USDA, acknowledging the manufacturing, production, and consumption to be in legal bounds.

The disclosed invention seeks to provide significant health benefits for users digesting mycoprotein capsules. For example, a user will digest and dissolve mycoprotein capsules at a faster rate in their digestive system than gelatin or cellulose capsules. Furthermore, the disclosed invention will provide quicker release times of drugs, medicine, supplements, vitamins, and lack thereof, into the body during digestion after being consumed.

The disclosed invention satisfies specific needs of a user from a shell designed to be hard, soft, and liquid, as well as being sealed, or left to remain non-sealed, with up to ten different size alternatives.

The disclosed invention seeks to give the user a new digestive method for drugs, medicine, supplements, vitamins, and lack thereof, which will best fit the users diagnosis, anatomy, philosophy, and financial means.

The practice of the disclosed invention seeks to reduce the cost of the capsule market by providing cleaner and cheaper energy security needed for pharmaceutical companies from less expenditures, and will provide safer laboratories without usage of hazardous materials such as rotting animal corpses to make gelatin capsules. The practice of the invention described will also provide implementations of water waste reductions that are currently excessive from creating genetically modified hypromellose and cellulose capsules.

The expressed production cycle of the disclosed invention seeks to utilize less time, space, and energy, while producing medicine to the world population quicker with a steadfast process of growing mycoprotein. Wherein, the world population will have more medicine available to the public, and in result, epidemic percentages will decrease.

An expressed function of the disclosed invention is to stabilize in moderate temperatures between 5° C. to 45° C. for successful inhabitability.

An expressed function of the disclosed invention is to preserve drugs, medicine, supplements, vitamins, and lack thereof.

An expressed function of the disclosed invention is to digest in the digestive system.

The disclosed invention seeks to prevent migration of a mycoprotein polymer based fill formulation migrating into or through a capsule shell for improved capsule integrity.

What is claimed is:

1. A fill formulation comprising an aqueous composition mixture, the method comprising;
(i) a mixture of a polymer and a fill component mixture that will migrate into or through a hard, soft, or liquid capsule shell; with (ii) a plasticizing film-forming component to seal or band the exterior shell of the capsule cap and body, thereby producing said mycoprotein capsule; having (iii) serviceable functions for conforming means of inhabiting, preserving, and digesting.

2. The fill formulation of claim 1, wherein the polymer is mycoprotein.

3. The method of claim 2, wherein at least least one mycoprotein polymer is selected from the group consisting of Fusarium venenatum A/35 or Fusarium venenatum PTA-2684 and mixtures thereof.

4. The fill formulation of claim 1, wherein at least one fill component is a plasticizing agent.

5. The method of claim 4, wherein at least one plasticizing agent is selected from the group consisting of acetyl tributyl citrate, acetyl triethyl citrate, acetylated monoglyceride, castor oil, coconut oil, dibutyl phthalate, dibutyl sebacate, diethyl phthalate, fructose, glucose, glucose syrup, glycerin, glycerine, non-crystalising solutions of sorbitol, polyethylene glycol (PEG 200-6000), polyglycerol, 2-propylene, propylene glycol, pullulan, sorbitol, triacetyl glycerine, tributyl citrate, triethyl citrate, xylitol, and mixtures thereof.

6. The fill formulation of claim 1, wherein at least one fill component is an aqueous solution.

7. The fill formulation of claim 6, wherein the aqueous solution consists of a preservative agent.

8. The method of claim 6, wherein at least one preservative agent is selected from the group consisting of benzoic acid, choline chloride, metal propyl esters of para hydroxy-benzoic, organic acid sulfur dioxide, propane acid, silicone dioxide, sodium bisulfite, sodium chloride, sodium metabisulfite, sorbic acid, sulfur dioxide, and mixtures thereof.

9. The fill formulation of claim 6, wherein the aqueous solution consists of a lubricant agent.

10. The method of claim 6, wherein at least one lubricant agent is selected from the group consisting of 2-butanol, hydroxypropyl-β-cyclodextrin, L-carvone, methyl-2-pentanone, methyl ethyl ketone, methyl n-propyl ketone, 2-pentanol, 2-propanol, sodium coco-sulfate, sodium lauryl sulfate, and mixtures thereof.

11. The fill formulation of claim 6, wherein the aqueous solution consists of purified H2O.

12. A capsule comprising the aqueous composition of the fill formulation wherein the fill formulation comprising the aqueous composition is comprised of any one of claims 1-11.

13. The capsule of claim 12, wherein the capsule is selected from a group consisting of hard, soft, and liquid capsules that are sizes “XXX”, “XXL”, “XX”, “XL”, “X”, “I”, “II”, “III”, “IV”, and “V”, and are universal sizes “000”, “00el”, “00”, “0el”, “0”, “1”, “2”, “3”, “4”, and “5”.

14. The capsule of claim 12, wherein the capsule is sealed or banded.

15. The method of claim 14, wherein the capsule is sealed or banded with pullulan used as an exterior plasticizer that is a yeast filial-forming agent.

16. The capsule of claim 12, wherein the primary component of the outer surface of the capsule shell comprises pullulan by weight.

17. The capsule of claim 12, wherein the primary component of the capsule composition comprises mycoprotein by weight.

18. The capsule of claim 12, wherein the primary component of the capsule composition comprises glycerol by weight.

19. The capsule of claim 12, wherein the primary component of the capsule composition comprises water by weight.

19. The capsule according to claim 12, wherein the dry aqueous composition of the shell comprises in weight percentage:

| | | |
|---|---|---|
| Polymer | Mycoprotein | 30%-56% |
| Plasticizer | Glycerol; (various) | 20%-30% |
| Solvent | H2O; (water) | 24%-40% |
| Excipients | Solution; (various) | 0.5%-1.5% |

20. A capsule comprising an inner surface and outer surface shell of claim 12, wherein the outer capsule comprises a fill formulation according to claim 1, and the inner capsule comprises at least one component selected from the group consisting of a placebo formulation, pharmaceutical dosage, therapeutic drug, nutritional agent, or dietary supplement, and combinations thereof.

21. A capsule comprising an inner surface and outer surface shell of claim 12, wherein the inner capsule comprises a component selected from the group consisting of a placebo formulation, pharmaceutical dosage, therapeutic drug, nutritional agent, dietary supplement, and combinations thereof, with the outer capsule comprising the inner capsule fill formulation according to claim 1.

22. The capsule of claim 20 or 21, wherein the inner surface and outer surface of the capsule each have a composition that are the same and that different with three serviceable functions.

23. A method of decreasing migration of the aqueous composition, comprising mixing at least one fill component with a mycoprotein polymer and loading the aqueous compositional matrix into or through a capsule shell to form hard, soft, or liquid capsules, according to claim 1.
